(19) United States
(12) Patent Application Publication
(10) Pub. No.: US 2026/0144960 A1
(43) Pub. Date: May 28, 2026

(54) CATHETER WITH TEMPERATURE SENSOR SYSTEM AND METHOD FOR PRODUCING THE SAME

(71) Applicants: Heraeus Medevio GmbH & Co. KG, Hanau (DE); Contract Medical International GmbH, Dresden (DE)

(72) Inventors: Ilias NIKOLAIDIS, Hanau (DE); Mario HEINTZE, Dresden (DE); Llewellyn Wallace GROENEVELD, Dresden (DE)

(21) Appl. No.: 19/395,812

(22) Filed: Nov. 20, 2025

(30) Foreign Application Priority Data
Nov. 25, 2024 (DE) 102024134585.3

(51) Int. Cl. A61M 25/00; A61B 18/00; A61B 18/14

(52) U.S. Cl. CPC A61M 25/0045; A61B 18/1492; A61M 25/0009; A61B 2018/00077; A61B 2018/00083; A61B 2018/00178; A61B 2018/00815; A61M 2205/0233; A61M 2205/0244; A61M 2205/3368; A61M 2207/00

(57) ABSTRACT

A catheter with a temperature sensor system having a catheter body including a hollow cylindrical inner liner having an inner liner outer surface, the inner liner extending in a longitudinal direction from a proximal catheter body end to a distal catheter body end of the catheter body, an outer insulation having an inner insulation surface, the outer insulation coaxially surrounding the inner liner, and a flexible circuit board comprising a plurality of electrical conductors, where the flexible circuit board is arranged between the inner liner outer surface and the inner insulation surface, and at least one electrode at the distal catheter body end, where the electrode is electrically conductively connected to at least one of the electrical conductors, wherein the flexible circuit board comprises at least one temperature sensor, where the temperature sensor is electrically conductively connected to at least one of the electrical conductors.

### CROSS-REFERENCE TO RELATED APPLICATION

This application claims priority pursuant to 35 U.S.C. 119(a) to German Patent Application No. 102024134585.3, filed Nov. 25, 2024, which application is incorporated herein by reference in its entirety.

### FIELD

The invention relates to a catheter with a temperature sensor system comprising a catheter body comprising a hollow cylindrical inner liner having an inner liner outer surface, the inner liner extending in a longitudinal direction from a proximal catheter body end to a distal catheter body end of the catheter body, an outer insulation having an inner insulation surface, the outer insulation coaxially surrounding the inner liner, and a flexible circuit board comprising a plurality of electrical conductors, wherein the flexible circuit board is arranged between the inner liner outer surface and the inner insulation surface, and at least one electrode at the distal catheter body end, wherein the electrode is electrically conductively connected to at least one of the electrical conductors, wherein the flexible circuit board comprises at least one temperature sensor, wherein the temperature sensor is electrically conductively connected to at least one of the electrical conductors.

The invention also relates to a method for producing such a catheter.

### BACKGROUND

Catheters have been used in medical procedures for many years. Among other things, catheters can be used in medical procedures to examine, diagnose and/or treat tissue while being used in areas of the body, such as blood vessels, that are not easily accessible without more invasive procedures. For example, catheters can be used to deliver electrical energy to selected locations in the human body, such as the heart, to specifically kill tissue, such as heart tissue. This is often referred to as “ablation”. Catheters can also be used to stimulate body tissue. During stimulation, the amount of energy transferred to the body tissue is usually lower than during ablation.

Another procedure, often called “mapping,” uses a catheter with sensing electrodes to monitor various forms of bioelectrical activity in the human body.

Catheters can therefore be used both to transmit (during “ablation” and “stimulation”) and to absorb (during “mapping”) energy to or from body tissue.

Regardless of the direction of energy flow, catheters often contain a plurality of electrodes, i.e., two or more, at a distal catheter body end region, wherein the distal catheter body end region often describes the end that is inserted into the patient. Typically, the catheter contains an equal number of electrodes at its proximal catheter body end region, which are in electrical contact with the electrodes at the distal catheter body end region. The electrodes at the proximal catheter body end region are used to connect electrical devices according to the desired areas of application of the catheter.

Temperature sensors are often used, in particular in the field of ablation catheters, to continuously monitor the temperature of the catheter, in particular its electrodes, as well as of the tissue to be treated. Accurate temperature control during ablation is crucial to ensure that the target tissue is heated sufficiently to achieve the desired therapeutic effect without negatively affecting adjacent healthy tissue. Excessively high temperatures can cause serious damage, including carbonization of tissue, which can lead to adverse effects such as the occurrence of arrhythmias. By using temperature sensors, energy delivery can be controlled precisely and in real time, effectively minimizing such risks and maximizing treatment effectiveness.

The temperature sensors, which are usually located near the distal end of the catheter, are usually electrically connected to the proximal end of the catheter via conductive wires or cables, as are the electrodes located there. Such wires or cables usually run in an inner lumen, a hollow space running axially through the catheter body of the catheter, and therefore either require the use of a larger catheter diameter or limit the possibilities of using the inner lumen for other purposes, for example to introduce additional devices such as a guidewire. Larger catheter diameters are disadvantageous because they could damage the patient's blood vessels. Furthermore, the temperature sensors are usually also located in the inner lumen of the catheter, which can limit the measurement accuracy for ablation taking place outside the catheter. On the other hand, placing the temperature sensor on the outer surface of the catheter results in an uneven outer diameter of the catheter and can lead to elevations or depressions, in particular sharp edges, which could cause damage to the patient's blood vessels. Furthermore, the installation of temperature sensors electrically contacted with wires or cables, whether in the inner lumen or on the outer surface of the catheter, is complex and therefore cost-intensive.

There is therefore a need in the market for catheters having an improved temperature sensor system.

### SUMMARY

The invention relates to a catheter with a temperature sensor system comprising a catheter body comprising a hollow cylindrical inner liner having an inner liner outer surface, the inner liner extending in a longitudinal direction from a proximal catheter body end to a distal catheter body end of the catheter body, an outer insulation having an inner insulation surface, the outer insulation coaxially surrounding the inner liner, and a flexible circuit board comprising a plurality of electrical conductors, wherein the flexible circuit board is arranged between the inner liner outer surface and the inner insulation surface, and at least one electrode at the distal catheter body end, wherein the electrode is electrically conductively connected to at least one of the electrical conductors, wherein the flexible circuit board comprises at least one temperature sensor, wherein the temperature sensor is electrically conductively connected to at least one of the electrical conductors. The invention also relates to a method for producing such a catheter.

The invention relates to a catheter with a temperature sensor system comprising a catheter body comprising a hollow cylindrical inner liner having an inner liner outer surface, the inner liner extending in a longitudinal direction from a proximal catheter body end to a distal catheter body end of the catheter body, an outer insulation having an inner insulation surface, the outer insulation coaxially surrounding the inner liner, and a flexible circuit board comprising a plurality of electrical conductors, wherein the flexible circuit board is arranged between the inner liner outer surface and the inner insulation surface, and at least one electrode at the distal catheter body end, wherein the electrode is electrically conductively connected to at least one of the electrical conductors, wherein the flexible circuit board comprises at least one temperature sensor, wherein the temperature sensor is electrically conductively connected to at least one of the electrical conductors. The invention also relates to a method for producing such a catheter.

One object of the present invention is to overcome, at least in part, one or more of the disadvantages resulting from the prior art.

It is an object of the present invention to provide a catheter with a temperature sensor system which comprises an inner lumen that is as clear as possible. In addition, the catheter should be able to be produced with the smallest possible outer diameter despite the temperature sensor system. The temperature sensor system should be installed in such a way that the temperature can be measured as precisely and quickly as possible. Furthermore, the catheter should be designed in such a way that it is as fail-safe and cost-effective to produce as possible.

It is a further object of the present invention to provide a method for producing such a catheter, which method is as simple, reliable and cost-effective as possible.

A contribution to the at least partial fulfillment of at least one of the aforementioned objects is made by the features of the independent claims. The dependent claims provide preferred embodiments that contribute to the at least partial fulfillment of at least one of the objects.

A first embodiment of the invention is a catheter with a temperature sensor system comprising a catheter body comprising a hollow cylindrical inner liner having an inner liner outer surface, the inner liner extending in a longitudinal direction from a proximal catheter body end to a distal catheter body end of the catheter body, an outer insulation having an inner insulation surface, the outer insulation coaxially surrounding the inner liner, and a flexible circuit board comprising a plurality of, i.e., at least two, electrical conductors, wherein the flexible circuit board is arranged between the inner liner outer surface and the inner insulation surface, and at least one electrode at the distal catheter body end, wherein the electrode is electrically conductively connected to at least one of the electrical conductors, characterized in that the flexible circuit board comprises at least one temperature sensor, wherein the temperature sensor is electrically conductively connected to at least one of the electrical conductors.

In a preferred embodiment of the catheter, the temperature sensor is a printed temperature sensor. This embodiment is a second embodiment of the invention, which is preferably dependent on the first embodiment of the invention.

In a preferred embodiment of the catheter, the flexible circuit board is arranged at least partially in a spiral shape in turns around the inner liner. This embodiment is a third embodiment of the invention, which is preferably dependent upon the first or second embodiment of the invention.

In a preferred embodiment of the catheter, the flexible circuit board is fixed between the inner liner and the outer insulation. This embodiment is a fourth embodiment of the invention, which is preferably dependent on one of the preceding embodiments of the invention.

In a preferred embodiment of the catheter, the inner liner and the outer insulation are at least partially integrally connected to one another. This embodiment is a fifth embodiment of the invention, which is preferably dependent on one of the preceding embodiments of the invention.

In a preferred embodiment of the catheter, the temperature sensor is designed as a meandering electrical conductor track. This embodiment is a sixth embodiment of the invention, which is preferably dependent on one of the preceding embodiments of the invention.

In a preferred embodiment of the catheter, the meandering electrical conductor track comprises a metal selected from the group consisting of silver, platinum, gold and, preferably, copper. This embodiment is a seventh embodiment of the invention, which is preferably dependent on the sixth embodiment of the invention.

In a preferred embodiment of the catheter, the temperature sensor is designed as a thermistor. This embodiment is an eighth embodiment of the invention, which is preferably dependent on one of the preceding embodiments of the invention.

In a preferred embodiment of the catheter, the thermistor is a printed thermistor. This embodiment is the ninth embodiment of the invention, which is preferably dependent on the eighth embodiment of the invention.

In a preferred embodiment of the catheter, the thermistor comprises a material selected from the group consisting of poly-3,4-ethylenedioxythiophene, polydimethylsiloxane, carbon nanotubes, graphene oxide, silicon carbide and graphene. This embodiment is a tenth embodiment of the invention, which is preferably dependent upon the eighth or ninth embodiment of the invention.

In a preferred embodiment of the catheter, the temperature sensor has a radial extension, or in other words a thickness, which is less than a radial extension, or in other words thickness, of the outer insulation. This embodiment is an eleventh embodiment of the invention, which preferably depends upon one of the preceding embodiments of the invention.

In a preferred embodiment of the catheter, the at least one temperature sensor is arranged at the same axial height of the catheter as the at least one electrode. This embodiment is a twelfth embodiment of the invention, which preferably depends upon one of the preceding embodiments of the invention.

A thirteenth embodiment of the invention relates to a method for producing a catheter with a temperature sensor system according to any of the preceding embodiments of the invention, comprising the method steps of:
a) providing a hollow cylindrical inner liner with a radially outer inner liner outer surface;
b) providing a flexible circuit board comprising a plurality of electrical conductors and comprising at least one temperature sensor, wherein the temperature sensor is electrically connected to at least one of the electrical conductors;
c) arranging the flexible circuit board on the inner liner outer surface;
d) sheathing the inner liner and the flexible circuit board with an outer insulation;
e) providing at least one electrode;
f) electrically contacting the at least one electrode with at least one of the electrical conductors of the flexible circuit board.

In a preferred embodiment of the method, the sheathing of the inner liner in method step d) comprises pulling at least one hollow cylindrical polymer tube onto the inner liner and the flexible circuit board, which tube, after being pulled on, undergoes a radial contraction by heating in order to form the outer insulation. This embodiment is a fourteenth embodiment of the invention, which is preferably dependent on the thirteenth embodiment of the invention.

In a preferred embodiment of the method, at least one contact opening is created in the outer insulation for electrically contacting the electrode in method step f) in order to selectively electrically contact the electrode with at least one electrical conductor of the flexible circuit board that is accessible via the contact opening, in particular accessible from the radial outside. This embodiment is a fifteenth embodiment of the invention, which is preferably dependent on the thirteenth or fourteenth embodiment of the invention.

With respect to the embodiments described herein, the elements of which “have,” or “comprise,” a particular feature (for example, a material), in principle, a further embodiment is always contemplated in which the relevant element consists solely of the feature, i.e., does not comprise any other constituents. The word “comprise” or “comprising” is used herein synonymously with the word “have” or “having.”

In one embodiment, if an element is denoted by the singular, an embodiment is also contemplated in which more than one such element is present. The use of a term for an element in the plural in principle also encompasses an embodiment in which only a single corresponding element is included.

Unless otherwise indicated or clearly excluded from the context, it is possible in principle, and is hereby clearly contemplated, that features of different embodiments may also be present in the other embodiments described herein. Likewise, it is contemplated in principle that all features described herein in connection with a method are also applicable to the products and devices described herein, and vice versa.

All such considered combinations are not explicitly listed in all instances, simply to keep the description brief. Technical solutions known to be equivalent to the features described herein are also intended in principle to be encompassed by the scope of the invention.

In the present description, specifications of ranges also contain the values specified as limits. A specification of the type “in the range from X to Y” with respect to a quantity A consequently means that A can take the values X, Y and values between X and Y. Ranges which are limited on one side, of the type “up to Y” for a size A, accordingly mean a value Y and less than Y.

Some of the features described are associated with the term “substantially.” The term “substantially” is to be understood in such a way that, under real conditions and manufacturing techniques, a mathematically exact interpretation of terms such as “superimposition,” “perpendicular,” “diameter” or “parallelism” can never be given exactly, but only within certain manufacturing error tolerances. For example, “substantially perpendicular axes” enclose an angle of 85 degrees to 95 degrees relative to one another, and “substantially equal volumes” comprise a variation of up to 5% by volume. For example, a “device consisting substantially of plastics” comprises a plastics content of ≥95 to ≤100% by weight. For example, a “substantially complete filling of a volume B” comprises a filling of ≥95 to ≤100% by volume of the total volume of B.

A first subject matter of the invention relates to a catheter with a temperature sensor system comprising
a catheter body comprising a hollow cylindrical inner liner having an inner liner outer surface, the inner liner extending in a longitudinal direction from a proximal catheter body end to a distal catheter body end of the catheter body,
an outer insulation having an inner insulation surface, the outer insulation coaxially surrounding the inner liner, and
a flexible circuit board comprising a plurality of electrical conductors, wherein the flexible circuit board is arranged between the inner liner outer surface and the inner insulation surface, and
at least one electrode at the distal catheter body end, wherein the electrode is electrically conductively connected to at least one of the electrical conductors, characterized in that the flexible circuit board comprises at least one temperature sensor, wherein the temperature sensor is electrically conductively connected to at least one of the electrical conductors.

The electrical conductor which is electrically conductively connected to the electrode is preferably a different electrical conductor than the electrical conductor which is electrically conductively connected to the temperature sensor.

A catheter comprises an elongate, tube-like catheter body which, depending on the application of the catheter, is equipped with an electrode or a plurality of electrodes, at least at a distal catheter body end, preferably both at the distal catheter body end and at a catheter body proximal end axially opposite the distal catheter body end. The electrodes at the proximal end of the catheter body are sometimes called connectors. In the present invention, the catheter body comprises a hollow cylindrical inner liner, a flexible circuit board and an outer insulation.

The catheter body, in particular the catheter body, comprises a hollow cylindrical inner liner with a radially outer inner liner outer surface. The inner liner extends axially from a proximal catheter body end to a distal catheter body end and preferably radially surrounds at least one inner lumen extending axially through the catheter body. The inner lumen is used to conduct and/or introduce various devices or fluids. For example, the inner lumen is used to insert a guide wire, which facilitates the insertion of the catheter into the patient.

The inner liner comprises an inner liner inner diameter which is, for example, in a range of 0.75 to 4 mm, and an inner liner outer diameter which is, for example, in a range of 1 to 4.5 mm. Depending on the application, the inner liner can comprise different lengths. For example, the inner liner comprises a length in a range from 30 cm to 200 cm.

The inner liner can be made of a variety of different materials, wherein the use of a flexible material or a flexible material combination is preferred. For example, the inner liner is made of a polymer that is selected from the group of silicones, polyolefins (e.g. polyethylene), polyether block amide (e.g. PEBAX©), polyurethanes, polyimides, polyamides, polyaryletherketones (e.g. polyetheretherketone), fluorinated polymers (e.g. selected from the group of ethylene tetrafluoroethylene, polytetrafluoroethylene, perfluoroalkoxyalkanes, polyvinylidene fluorides, fluorinated ethylene propylene and mixtures thereof) and mixtures thereof. According to a preferred embodiment, the inner liner comprises a polymer that is selected from the group of silicones, polyolefins (e.g. polyethylene), polyether block amide (e.g. PEBAX©), polyurethanes, polyimides, polyamides, polyaryletherketone (e.g. polyetheretherketone), fluorinated polymers (e.g. selected from the group of ethylene tetrafluoroethylene, polytetrafluoroethylene, perfluoroalkoxyalkanes, polyvinylidene fluorides, fluorinated ethylene propylene and mixtures thereof) and mixtures thereof.

An outer insulation having an inner insulation surface is arranged coaxially around the inner liner, the inner insulation surface facing the outer liner surface. The outer insulation is preferably the radially outer layer of the catheter, in particular of the catheter body, and serves in particular to electrically insulate the catheter from the outside and to mechanically protect it. Preferably, the outer insulation has an outer diameter in a range of 200 μm to 5000 μm, more preferably 300 μm to 3000 μm and most preferably 500 μm to 1500 μm. In an exemplary embodiment, the outer diameter of the outer insulation is about 700 μm. It is understood that the outer diameter of the outer insulation can also correspond to the outer diameter of the catheter. The outer insulation preferably has a wall thickness in the range of 2 μm to 300 μm, particularly preferably from 5 μm to 150 μm, and most preferably from 20 μm to 100 μm. According to a preferred embodiment of the present invention, the outer insulation has an outer diameter in a range of 300 μm to 3000 μm, particularly preferably from 500 μm to 1500 μm, and a wall thickness in a range of 5 μm to 150 μm, particularly preferably from 20 μm to 100 μm. In an exemplary embodiment, the wall thickness is approximately 60 μm. The outer insulation preferably consists of a polymer that is selected from the group of silicones, polyolefins (e.g. polyethylene), polyether block amide (e.g. PEBAX©), polyurethanes, polyimides, polyamides, polyaryletherketones (e.g. polyetheretherketone), fluorinated polymers (e.g. selected from the group of ethylene tetrafluoroethylene, polytetrafluoroethylene, perfluoroalkoxyalkanes, polyvinylidene fluorides, fluorinated ethylene propylene and mixtures thereof) and mixtures thereof. According to a preferred embodiment, the outer insulation comprises a polymer that is selected from the group of silicones, polyolefins (e.g., polyethylene), polyurethanes, polyimides, polyamides, polyaryletherketone (e.g., polyetheretherketone), fluorinated polymers (e.g., selected from the group of ethylene tetrafluoroethylene, polytetrafluoroethylene, perfluoroalkoxyalkanes, polyvinylidene fluorides, fluorinated ethylene propylene, and mixtures thereof), and mixtures thereof, and has an outer diameter in the range of 300 to 3000 μm.

A flexible circuit board is arranged radially between the inner liner and the outer insulation, i.e., between the inner insulation surface and the outer inner liner surface. A flexible circuit board, frequently also called flex circuit ribbon, is a circuit board comprising or consisting of flexible, bendable materials that allow the circuit board to be bent or folded or wrapped into different shapes. Typically, the structure of a typical flexible circuit board comprises several layers. The core element is one or more electrically conductive layers, for example made of a metal foil or a printed metal layer preferably comprising copper or consisting of copper, which can contain a specific pattern, depending on the type of application. This layer or a conductor track within this layer acts as the electrical conductor of the flexible circuit board. The electrically conductive layer(s) is/are usually embedded between electrically insulating layers made of flexible insulation material such as polyimides, which can also provide mechanical protection. For example, the flexible circuit board has a plurality of, i.e., at least two, metallic conductor tracks as electrical conductors, wherein the electrical conductors are embedded in layers of a polyimide.

Flexible circuit boards offer advantages when used in catheters, in particular due to their small space requirement, low weight and simplified installation. In particular, installing individual conductive wires, in particular through the lumen of the inner liner, is unnecessary due to the use of a flexible circuit board.

The flexible circuit board of the catheter body according to the invention comprises at least two electrical conductors. For example, the flexible circuit board comprises two to 20 electrical conductors, each of which can be electrically connected to one or more electrodes at least partially. Preferably, the electrical conductors are electrically isolated from each other. More preferably, the flexible circuit board comprises at least two insulating layers which electrically insulate the at least two electrical conductors from the outside at least over long sections of the relevant electrical conductors. The flexible circuit board may comprise contact surfaces at certain positions which make the electrical conductor electrically contactable from the outside without the need to remove part of the insulating layer beforehand.

By placing the flexible circuit board between the inner liner and the outer insulation, the inner lumen of the catheter body remains free for other uses, which expands the possible applications of the catheter. Furthermore, a catheter body with the smallest possible outer diameter can be realized in this way. In addition, the flexible circuit board is more protected against damage because any devices pushed through the inner lumen, such as a guide wire, do not come into direct contact with the flexible circuit board.

The catheter has at least one electrode. The exact number of electrodes depends on the use of the catheter. For example, the catheter has 1 to 8 electrodes at the distal catheter body end, each of which is electrically connected to at least one, preferably exactly one, electrical conductor of the flexible circuit board.

Preferably, the at least one electrode is a ring electrode. Further preferably, all electrodes are ring electrodes.

The electrode can comprise a plurality of different materials or consist of different materials. Preferably, the electrode comprises a metal that is selected from the group consisting of platinum, iridium, tantalum, palladium, titanium, iron, gold, molybdenum, niobium, tungsten, nickel, chromium, cobalt, steel, nitinol, alloys of any of these metals, and composites of any of these metals. Stainless steel is suitable as an electrode, for example stainless steel AISI 316L, stainless steel AISI 301 or stainless steel AISI 304. Platinum and platinum alloys such as Pt/Ir 10 or Pt/Ir 20 or nickel-cobalt alloys such as MP35N are also suitable as electrodes.

The choice of metal for the electrodes (and for the electrical conductors) can depend on the use of the catheter according to the invention. However, it should be understood that the application of the catheter is not limited by the use of a particular metal.

The electrode can also comprise a coating. Suitable coatings are metal nitrides such as TiN, metal oxides such as IrO₂ or conductive polymers such as poly-3,4-ethylenedioxythiophene (PEDOT). The surface of the electrode can also be surface-structured, e.g., laser-structured.

The electrode, preferably a ring electrode, can have an outer diameter in the range of 200 to 5000 μm, in one embodiment in the range of 300 to 3000 μm, and in one embodiment in the range of 500 to 1500 μm. The electrode can have a wall thickness in the range of 10 to 200 μm, in one embodiment 10 to 100 μm, and in one embodiment 30 to 70 μm. Furthermore, the electrode can have a length in the range of 200 to 5000 μm, in one embodiment 300 to 3000 μm, and in one embodiment in the range of 500 to 1500 μm. According to one embodiment, the electrode has an outer diameter in the range of 300 to 3000 μm and in one embodiment in the range of 500 to 1500 μm, a wall thickness in the range of 10 to 100 μm and in one embodiment of 30 to 70 μm, and a length in the range of 300 to 3000 μm and in one embodiment in the range of 500 to 1500 μm.

The flexible circuit board of the catheter has at least one temperature sensor. The at least one temperature sensor is preferably an integral part of the flexible circuit board, i.e., preferably not an additional, separate component which is mounted on the flexible circuit board.

The at least one temperature sensor serves to monitor the ambient temperature and/or the temperature of the electrodes of the catheter, in particular during the proper use of the catheter, in particular during ablation of patient tissue.

For this purpose, the temperature sensor has a sensor element which can be electrically contacted with a signal processing unit via the electrical connection to one of the electrical conductors of the flexible circuit board. The signal processing unit is used to evaluate the signals from the temperature sensor, preferably in real time, and can thus display the temperature and its changes to the user of the catheter. The temperature sensor, together with the electrical conductor electrically connected to the temperature sensor, forms the temperature sensor system of the catheter.

The flexible circuit board thus preferably has at least two electrical conductors: one of the at least two electrical conductors serve to electrically contact the at least one electrode of the catheter, the second electrical conductor serves to electrically contact the at least one temperature sensor of the catheter.

The exact number of temperature sensors depends on the use of the catheter. For example, the catheter comprises 1 to 100 temperature sensors, in particular at the distal catheter body end in the region of the electrodes. In one embodiment, the catheter has as many temperature sensors as electrodes, so that each of the temperature sensors can measure the temperature in the region of one of the electrodes.

The temperature sensor, like the rest of the flexible circuit board, is thus arranged between the inner liner and the outer insulation. In other words, the at least one temperature sensor is arranged in the wall of the catheter body. This brings the temperature sensor closer to the patient tissue and the electrodes than is the case with temperature sensors located within the inner lumen, where they are usually located. This arrangement allows for a more precise and faster measurement of the temperature, in particular of the patient tissue, because there is less “material” of the catheter body between the sensor and the patient tissue or the catheter electrode.

Furthermore, the assembly of such a catheter is simplified compared to the classic electrical contacting of a temperature sensor by means of wires or cables, because the sensor can be provided in an already wired state on the flexible circuit board.

The temperature sensor, in particular its sensor element, can be implemented in different ways as part of the flexible circuit board. For example, different types of metal foils, for example in stamped form, can be used to construct the temperature sensor.

A preferred embodiment of the catheter is characterized in that the temperature sensor is a printed temperature sensor. This refers to a temperature sensor which is constructed using liquid or pasty compositions. Depending on their viscosity, these compositions can be used to construct the temperature sensor by means of different printing methods. Low-viscosity to liquid compositions can be used, for example by means of ink jet printing, while higher-viscosity compositions, such as thick film pastes, can be used, for example by means of stencil or screen printing, to construct the temperature sensor. Another possibility for creating printed temperature sensors is the use of 3D printing. Printed temperature sensors have a simple design with few or no moving components, are cost-effective to produce and can be manufactured in particular with a very low installation height, for example in a range between 0.01 μm and 20 μm. In particular, the preferred low overall height (in radial extension relative to the catheter body) in a range between 0.01 μm and 20 μm is advantageous for producing a catheter body having the smallest possible outer diameter.

Depending on the type of liquid or pasty composition, the construction of the printed temperature sensor may comprise a curing process, in particular a thermal curing process.

The flexible circuit board can be arranged between the inner liner and the outer insulation in different ways. For example, the flexible circuit board can run straight along the longitudinal axis of the catheter, in particular of the catheter body. This arrangement is particularly easy to implement in terms of manufacturing technology.

A preferred embodiment of the catheter is characterized in that the flexible circuit board is arranged in a spiral shape in turns around the inner liner at least partially, preferably substantially completely over the entire length of the flexible circuit board. The flexible circuit board therefore extends preferably axially in spiral turns between the inner liner and the outer insulation of the catheter body, preferably along substantially the entire length of the catheter body.

The arrangement in spiral turns has the advantage that the flexible circuit board can adapt particularly well to bends in the catheter body, which can reduce the risk of damage to the flexible circuit board when using the catheter.

To minimize or even completely prevent unintentional displacement, in particular in the axial direction, of the circuit board during use of the catheter, a preferred embodiment of the catheter is characterized in that the flexible circuit board is fixed between the inner liner and the outer insulation. This can be caused, for example, by the flexible circuit board being firmly jammed between the inner liner and the outer insulation. In a further embodiment, the flexible circuit board can be attached to the inner liner, the outer insulation or to the inner liner and the outer insulation by means of an adhesive, such as an adhesive tape, in particular a double-sided adhesive tape, to fix it.

In order to provide a catheter body that is as mechanically stable as possible, a preferred embodiment of the catheter is characterized in that the inner liner and the outer insulation are at least partially integrally connected to one another. Preferably, the inner liner and the outer insulation are integrally connected to one another in the spaces between the preferred spiral turns of the flexible circuit board, for example by fusing. This has the advantage, for example, that the flexible circuit board is at least partially spatially secured by this integral bond and cannot, for example, slip between the inner liner and the outer insulation, which could be the case with layers not connected to each other. To facilitate the production of the catheter body, the flexible circuit board can be wrapped around the inner liner as desired and secured to the inner liner outer surface using an adhesive such as an adhesive or an adhesive strip, for example a double-sided adhesive tape, before the outer insulation in the form of a hollow cylindrical hose or tube is pulled on, or in other words, pulled over, the inner liner and the flexible circuit board. A temperature treatment afterwards can form the integral bond between the inner liner and the outer insulation in the spaces between the turns of the flexible circuit board. Preferably, the flexible circuit board itself does not comprise a layer which, due to this temperature treatment itself, forms an integral bond with the inner liner or the outer insulation. For example, the flexible circuit board comprises, at least on the outside, only polymer layers made of a polyimide.

The temperature sensor, in particular in the form of a printed temperature sensor, can use different technologies to measure temperature.

A preferred embodiment of the catheter is characterized in that the temperature sensor, in particular its sensor element, is designed as a meandering electrical conductor track. Such a temperature sensor has meandering electrically conductive paths, i.e., repeating turns, bends or curves in a, preferably continuous, pattern made of an electrically conductive material, wherein a resistance of this conductor path, which changes with temperature, is determined for temperature measurement. The meandering arrangement of the electrically conductive track serves to maximize its length in a limited space. Such a temperature sensor can often be produced cost-effectively and can also be easily applied to larger areas, preferably as a printed temperature sensor.

Preferably, the electrical conductor track has a specific resistance value of 10 mOhms/square/mil or lower.

The meandering electrical conductor track of the temperature sensor can consist of different electrically conductive materials or material combinations.

A preferred embodiment of the catheter is characterized in that the meandering electrical conductor track comprises at least one metal selected from the group consisting of silver, platinum, gold and, preferably, copper. Preferably, the conductor track consists of one of these metals or a combination of these metals.

Copper is preferred because the electrical conductors of the flexible circuit board preferably also comprise copper or, preferably, consist of copper, so that the electrical conductors and the meandering electrical conductor track can be formed in the same way, preferably even in the same method step, for example a printing process of a thick film paste.

A preferred embodiment of the catheter is characterized in that the temperature sensor is formed as a thermistor. Preferably, the thermistor is manufactured using a thermistor paste. The temperature sensor is therefore a temperature sensor made of a hardened thermistor paste.

Such temperature sensors preferably contain a mixture of electrically highly conductive materials, preferably silver or copper, as well as special materials, such as certain polymers, whose resistance properties are subject to a strong temperature dependence. In contrast to temperature sensors made of meandering electrically conductive traces, there is usually no need to maximize the length of the electrical resistor, as it is inherently more sensitive to temperature changes. Thermistors, their materials and their structure are familiar to a person skilled in the art.

The specific resistance value of the thermistor is preferably in the range of 100 mOhms/square/mil or higher.

A preferred embodiment of the catheter is characterized in that the thermistor is a printed thermistor. This type of thermistor is preferably manufactured using a thermistor paste by means of stencil printing or screen printing.

A preferred embodiment of the catheter is characterized in that the thermistor comprises at least one material selected from the group consisting of poly-3,4-ethylenedioxythiophene, polydimethylsiloxane, carbon nanotubes, graphene oxide, silicon carbide and graphene. More preferably, the thermistor comprises a conductive metal selected from the group consisting of silver, platinum, gold and copper.

A preferred embodiment of the catheter is characterized in that the temperature sensor has a radial extension, in particular an extension perpendicular to the longitudinal axis of the catheter body, or in other words a “thickness” which is less than a radial extension of the outer insulation. Because the temperature sensor is arranged between the inner liner and the outer insulation, such a temperature sensor allows the production of a catheter body with an outer diameter that is as uniform as possible. The catheter body therefore has as few elevations or depressions as possible, or none, which makes it easier to insert into a patient's blood vessels and minimizes the risk of vascular damage.

Preferably, the temperature sensor has a radial extension in a range between 0.01 μm and 20 μm.

The arrangement of the at least one temperature sensor relative to the at least one electrode can be freely selected depending on the application. For example, a temperature sensor can be arranged axially between two axially adjacent electrodes.

A preferred embodiment of the catheter is characterized in that the at least one temperature sensor is arranged at the same axial height of the catheter, in particular of the catheter body, as the at least one electrode. In this embodiment, the temperature sensor is thus arranged radially under the electrode, which allows a fast and precise temperature measurement of the electrode or, during use of the catheter, of the body tissue surrounding the electrode. The temperature sensor positioned in this way is arranged closer to the electrode than would be the case, for example, with a temperature sensor arranged in the inner lumen of the catheter body.

Preferably, the electrical contacting of the electrode with an electrical conductor of the flexible circuit board takes place spatially separated from the temperature measurement by means of the temperature sensor of the flexible circuit board. Preferably, the electrode is a ring electrode, so that the electrical contacting of the ring electrode with an electrical conductor of the flexible circuit board at a circumferential position of the ring electrode and the temperature measurement by means of the temperature sensor at a further circumferential position of the ring electrode, and thus spatially separated from each other, can be realized.

A further subject matter of the invention relates to a method for producing a catheter with a temperature sensor system according to any of the preceding embodiments of the invention, comprising the method steps of:
a) providing a hollow cylindrical inner liner with a radially outer inner liner outer surface;
b) providing a flexible circuit board comprising a plurality of electrical conductors and comprising at least one temperature sensor, wherein the temperature sensor is electrically connected to at least one of the electrical conductors;
c) arranging the flexible circuit board on the inner liner outer surface;
d) sheathing the inner liner and the flexible circuit board with an outer insulation;
e) providing at least one electrode;
f) electrically contacting the at least one electrode with at least one of the electrical conductors of the flexible circuit board.

To avoid repetition, reference is made to the previous description for a description of the features of the individual elements of the catheter.

In a method step a), the hollow cylindrical inner liner with a radially outer inner liner outer surface is provided.

In a method step b) the flexible circuit board is provided. Preferably, the provision comprises a printing process by which the temperature sensor is applied to the flexible circuit board. More preferably, this printing process is carried out with a thick film conductive paste, such as a copper paste for forming a meandering conductor track, or a thermistor paste containing, for example, silver and carbon nanotubes for forming a thermistor.

In a method step c), the inner liner outer surface is arranged. For example, the flexible circuit board is placed axially on the inner liner, in particular the inner liner outer surface. In a preferred embodiment of the method, the flexible circuit board is wound spirally in turns around the inner liner, preferably over the entire length of the flexible circuit board.

To simplify assembly and/or improve the stability of the finished catheter, the flexible circuit board may be fixed to the inner liner, for example by means of an adhesive or an adhesive tape, for example a double-sided adhesive tape. This fixation preferably remains on the catheter so that the flexible circuit board is fixed in the finished catheter between the inner liner and the outer insulation.

In a method step d), the inner liner and the flexible circuit board arranged on it are sheathed with the outer insulation. The sheathing can be carried out in different ways. For example, the sheathing may comprise coaxially extruding the outer insulation around the inner liner and the flexible circuit board.

A preferred embodiment of the method is characterized in that the sheathing of the inner liner, and at the same time also of the flexible circuit board, in method step d) comprises pulling a hollow cylindrical polymer tube, preferably made of a silicone, polyolefin (e.g., polyethylene), polyether block amide (e.g., PEBAX©), polyurethane, polyimide, polyamide, polyaryletherketone (e.g., polyetheretherketone), fluorinated polymer (e.g., selected from the group of ethylenetetrafluoroethylene, polytetrafluoroethylene, perfluoroalkoxyalkanes, polyvinylidene fluorides, fluorinated ethylenepropylene and mixtures thereof) and mixtures thereof, onto the inner liner and the flexible circuit board, wherein the polymer tube undergoes a radial contraction by heating after pulling on in order to form the outer insulation. In this embodiment, a polymer tube which contracts radially under thermal influence and has an inner diameter which corresponds at least to the outer diameter of the inner liner and the flexible circuit board arranged there, is pushed onto the inner liner. Once the polymer tube has reached the desired position, it is heated, whereupon the polymer tube contracts radially and forms the outer insulation of the catheter, in particular of the catheter body. The radial contraction is preferably carried out at a temperature ranging between 120° C. and 180° C.

In a method step e), at least one electrode is provided. The exact number of electrodes depends on the use of the catheter.

Preferably, the electrodes are ring electrodes. Further preferably, all provided electrodes are ring electrodes.

The electrode can comprise a plurality of different materials or consist of different materials. Preferably, the electrode comprises a metal that is selected from the group consisting of platinum, iridium, tantalum, palladium, titanium, iron, gold, molybdenum, niobium, tungsten, nickel, chromium, cobalt, steel, nitinol, alloys of any of these metals, and composites of any of these metals. Stainless steel is suitable as an electrode, for example stainless steel AISI 316L, stainless steel AISI 301 or stainless steel AISI 304. Platinum and platinum alloys such as Pt/Ir 10 or Pt/Ir 20 or nickel-cobalt alloys such as MP35N are also suitable as electrodes.

The choice of metal for the electrodes (and for the electrical conductors) can depend on the use of the catheter according to the invention. However, the application of the catheter is not limited by the use of a particular metal.

The electrode can also comprise a coating. Suitable coatings are metal nitrides such as TiN, metal oxides such as IrO₂ or conductive polymers. The surface of the electrode can also be surface-structured, e.g., laser-structured.

The electrode, preferably a ring electrode, can have an outer diameter in the range of 200 to 5000 μm, in one embodiment in the range of 300 to 3000 μm, and in one embodiment in the range of 500 to 1500 μm. The electrode can have a wall thickness in the range of 10 to 200 μm, in one embodiment 10 to 100 μm, and in one embodiment 30 to 70 μm. Furthermore, the electrode can have a length in the range of 200 to 5000 μm, in one embodiment 300 to 3000 μm, and in one embodiment in the range of 500 to 1500 μm. According to one embodiment, the electrode has an outer diameter in the range of 300 to 3000 μm and in one embodiment in the range of 500 to 1500 μm, a wall thickness in the range of 10 to 100 μm and in one embodiment of 30 to 70 μm, and a length in the range of 300 to 3000 μm and in one embodiment in the range of 500 to 1500 μm.

In a method step f), the at least one electrode is electrically contacted with at least one of the electrical conductors of the flexible circuit board. For example, this electrical contact can be made by purely physical contact. In further embodiments, the electrical contacting may comprise a contact means, such as a conductive adhesive or a conductive paste, which provides an electrical contact and at the same time a mechanical fixation. Furthermore, the mechanical stability of the electrical contact can be achieved by means of a welding process, for example by means of laser welding. Preferably, the electrode is a ring electrode and the electrical contacting comprises radial compression of the ring electrode toward the inner liner.

The method steps d) as well as e) and f) can be carried out in a different order.

If method step d) is carried out before method step f), it may be necessary to remove parts of the outer insulation to form an electrical contact between the electrode and the electrical conductor.

A preferred embodiment of the method is characterized in that, for electrically contacting the electrode, preferably in the form of a ring electrode, at least one contact opening is created in the outer insulation in method step f) to selectively electrically contact the electrode with at least one electrical conductor of the flexible circuit board that is accessible via the contact opening. To create the contact opening, part of the outer insulation and, if necessary, part of the insulating layer of the flexible circuit board is removed at the corresponding position so that the at least one electrical conductor is exposed radially from the outside via the contact opening to be electrically contactable. The removal of parts of the insulating layer of the flexible circuit board is in particular necessary if the flexible circuit board does not already comprise an accessible electrical conductor at the position of the contact opening or has a contact surface via which the electrical conductor can be electrically contacted from the outside without removing parts of the insulating layer. At the position of the contact opening, an electrode can be selectively, or in other words specifically, electrically contacted with the electrical conductor over the course of the subsequent method. If the catheter body is to comprise more than one electrode, in particular at the distal catheter body end, it is preferred that a separate contact opening is created for each of these electrodes, wherein one of the electrical conductors of the flexible circuit board is selectively electrically contacted via each of these contact openings. For this purpose, the flexible circuit board preferably comprises at least as many electrical conductors as electrodes are to be attached to the distal catheter body end.

The contact opening can be created in different ways. For example, the contact opening can be created by cutting, punching or, preferably, by laser ablation.

In further embodiments, the outer insulation is selectively sheathed around the inner liner and the flexible circuit board, so that the positions at which an electrode is to be electrically contacted with at least one electrical conductor of the flexible circuit board remain free of the outer insulation. This can be achieved, for example, by using two or more polymer tubes that contract radially when heated and which are pushed onto the inner liner at an axial distance from each other and then heated. In these embodiments, the gap between two of these axially adjacent polymer tubes can be used for insertion of an electrode, preferably a ring electrode. Preferably, the width of the gap corresponds to the width of the electrode used there.

In further embodiments of the method, a contact means can be used. The contact means serves to electrically contact the electrical conductor of the flexible circuit board with an electrode via the contact opening. The contact means is a conductive material, at the latest after a temperature treatment, which can simultaneously, at least after a temperature treatment, mechanically secure the electrical conductor to the electrode. The contact means also serves to bridge the depth of the contact opening, which corresponds at least to the thickness of the outer insulation, so that neither the electrical conductor nor the electrode to be connected thereto have to overcome the radial depth of the contact opening alone. Without contact means, for example, the electrical conductor would have to be at least partially pulled radially outwards, and/or the electrode would have to have a radially inwardly directed projection which can overcome at least significant parts of the depth of the contact opening.

The contact means can be designed in different ways. The contact means is preferably a solder, for example in the form of a solder paste, a conductive paste or a sintering paste. This type of contact means allows an electrical connection between the electrical conductor and the electrode and, after a temperature treatment, can simultaneously serve as a mechanical fastening agent between the electrical conductor and the electrode.

The properties and features disclosed in the description may be essential for various embodiments of the invention, both separately and in any combination with one another. The properties and features disclosed for the catheter are also disclosed for the method, and vice versa.

### BRIEF DESCRIPTION OF THE DRAWINGS

The invention is further illustrated by way of example below by means of figures. The invention is not limited to the figures.

In the figures:

FIG. 1 shows a schematic longitudinal section of a detail of an exemplary catheter;

FIG. 2 shows a schematic cross section of the catheter from FIG. 1; and,

FIG. 3 shows an example of a flow chart of a method for producing a catheter.

### DETAILED DESCRIPTION OF THE INVENTION

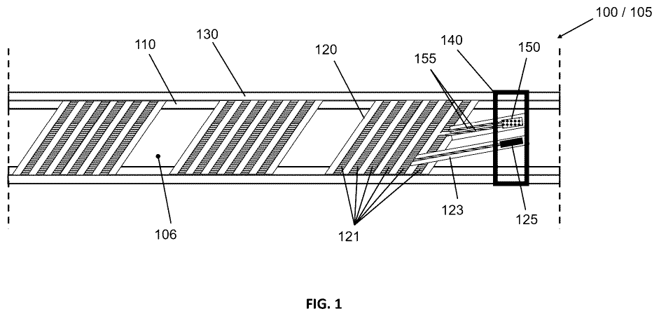

FIG. 1 shows a detail of an exemplary catheter 100, in particular a detail of a catheter body 105 of the catheter 100, in a schematic longitudinal section. The catheter 100 has a hollow cylindrical inner liner 110 which encloses an inner lumen 106 of the catheter 100. An outer insulation 130 is arranged coaxially around the inner liner 110. A flexible circuit board 120 is arranged in spiral turns between the inner liner 110 and the outer insulation 130 (shown in plan view for clarity). The inner liner 110 and the outer insulation 130 are integrally connected to one another between the turns of the flexible circuit board 130, so that the flexible circuit board 120 is fixed between the inner liner 110 and the outer insulation 130 and is protected against slipping, in the axial direction.

In the embodiment shown, the flexible circuit board 130 comprises six electrical conductors 121 that are insulated from one another. In further embodiments not shown, the flexible conductor 130 may comprise up to twenty or more electrical conductors.

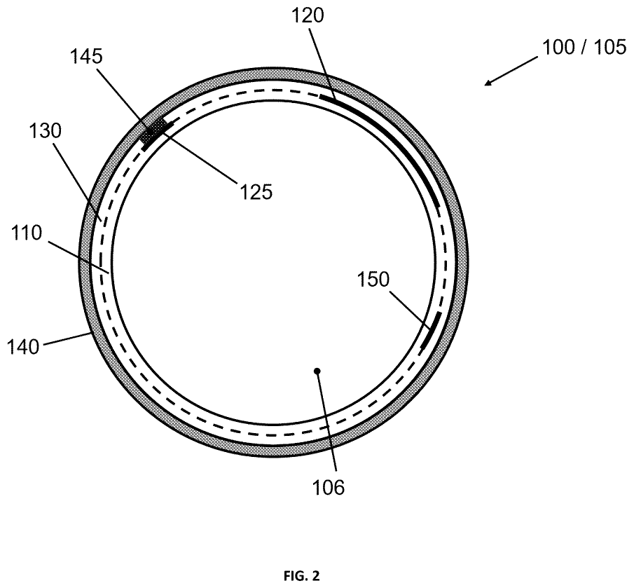

In the portion shown, the catheter 100 has an electrode 140 in the form of a ring electrode (position of the ring electrode is indicated by the black-bordered rectangle; for further details, reference is also made to FIG. 2).

The electrode 140 is electrically connected to one of the electrical conductors 121 of the flexible circuit board, namely the second one from the right. For this purpose, an electrically conductive bridge, which is electrically insulated from the other electrical conductors, was branched off in the direction of the electrode 140, which ends in a contact surface 125, via which the electrical contact with the electrode 140 is realized (shown in plan view for better clarity; for further details see FIG. 2).

Furthermore, the flexible circuit board 120 has a temperature sensor 150, for example in the form of a meandering conductor or a printed thermistor, which is electrically connected to one of the electrical conductors 121, namely the electrical conductor 121 on the far right, of the flexible circuit board 120 (shown in plan view for the sake of better clarity). For this purpose, two outwardly insulated electrically conductive paths 155 branch off from the electrical conductor 121 on the far right to the temperature sensor 150.

In the embodiment shown, the temperature sensor 150 is arranged at the same axial height as the electrode 140, which allows the smallest possible spacing between these two elements and thus the most precise and rapid temperature measurement of the electrode 140 and the patient tissue surrounding the electrode 140 when using the catheter 100.

FIG. 2 shows the catheter 100, in particular the catheter body 105 of the catheter 100, from FIG. 1 in a schematic cross section at the axial height of the electrode 140. The inner lumen 106 can be seen coaxially surrounded by the inner liner 110, which in turn is coaxially surrounded by the outer insulation 130 (demarcated by a dashed line), which in turn is coaxially surrounded by the electrode 140 in the form of a ring electrode. The flexible circuit board 120 is arranged between the inner liner 110 and the outer insulation 130. The flexible circuit board 120 has a radial extension, or in other words a thickness, which is less than a radial extension of the outer insulation 120, which provides a catheter 100, in particular a catheter body 105, with an outer diameter that is as uniform as possible.

In cross section, it is clearly visible that in the embodiment shown the main part of the flexible circuit board 120 (directly provided with reference numeral 120) is arranged spatially spaced apart from the contact surface 125 as well as spatially spaced apart from the temperature sensor 150 between the inner liner 110 and the outer insulation 130. The temperature sensor 150 is arranged almost on the opposite side of the contact surface 125.

In the cross section, it can be seen that a contact opening 145 filled with a contact means was created in the outer insulation for electrically contacting the contact surface 125 with the electrode 140. The contacting means is an electrically conductive medium, in the embodiment shown a conductive adhesive, which bridges the distance between the electrode 140 and the contact surface 125.

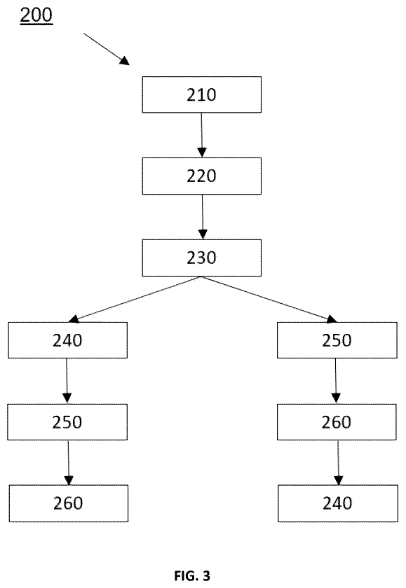

FIG. 3 shows a flow chart of an exemplary method 200 for producing a catheter 100 comprising the method steps 210 to 260.

In a method step 210, a hollow cylindrical inner liner 110 with a radially outer inner liner outer surface is provided.

In a method step 220, a flexible circuit board comprising a plurality of electrical conductors 121 and comprising at least one temperature sensor 150 is provided. The temperature sensor 150 is electrically connected to at least one of the electrical conductors 121, preferably to exactly one of the electrical conductors 121. Preferably, the temperature sensor 150 has been applied to the flexible circuit board 120 by a printing method such as stencil printing, screen printing or inkjet printing. The temperature sensor 140 is preferably an integral part of the flexible circuit board 120.

In a method step 230, the flexible circuit board 120 is arranged on the inner liner outer surface of the inner liner 110. In a preferred embodiment of the method 200, the flexible circuit board 120 is spirally wound in turns around the inner liner 110, preferably over the entire length of the flexible circuit board 110.

After method step 230, the method 200 can be carried out in different ways, two of which are illustrated here as examples.

In the first method (left path), after method step 230, a method step 240 takes place, which involves sheathing the inner liner 110 and the flexible circuit board 120 arranged thereon with an outer insulation 130. Preferably, the sheathing of the inner liner 110, and simultaneously also of the flexible circuit board 120, comprises pulling a hollow cylindrical polymer tube, preferably made of a polyether block amide, onto the inner liner 110 and the flexible circuit board 120, wherein the polymer tube undergoes a radial contraction by heating after pulling on in order to form the outer insulation 130. In this embodiment, a polymer tube which contracts radially under thermal influence and has an inner diameter which corresponds at least to the outer diameter of the inner liner and the flexible circuit board arranged there, is pushed onto the inner liner 110. Once the polymer tube has reached the desired position, it is heated, whereupon the polymer tube contracts radially and forms the outer insulation 130 of the catheter 100, in particular of the catheter body 105. In this embodiment of the method, in order to electrically contact the flexible circuit board 120, in particular its electrical conductor 121, with an electrode 140, it is therefore necessary to at least partially remove the outer insulation 130 while exposing the corresponding electrical conductor 121.

In a method step 250, at least one electrode 140 is provided, preferably in the form of a ring electrode. Preferably, all electrodes 140 provided are ring electrodes.

In a method step 260, the at least one electrode 140 is electrically contacted with at least one of the electrical conductors 121 of the flexible circuit board 120. For example, this electrical contact can be made by purely physical contact. In further embodiments, the electrical contacting may comprise a contact means, such as a conductive adhesive or a conductive paste, which provides an electrical contact and at the same time a mechanical fixation. Furthermore, the mechanical stability of the electrical contact can be achieved by means of a welding process, for example by means of laser welding. Preferably, the electrode 140 is a ring electrode and the electrical contacting comprises radially compressing the ring electrode toward the inner liner 110.

As already mentioned, electrical contacting in method step 260 requires removal of parts of the outer insulation 130 for the first procedure. For this purpose, a contact opening is created in the outer insulation 130 in order to selectively electrically contact the electrode 140 with at least one electrical conductor 121 of the flexible circuit board 120 that is accessible via the contact opening. To create the contact opening, part of the outer insulation 130 and, if necessary, part of the insulating layer of the flexible circuit board 120 is removed at the corresponding position so that the at least one electrical conductor 121 is exposed radially from the outside via the contact opening to be electrically contactable.

In a second procedure, the further method step 240 as well as 250 and 260 are carried out in a different order. Here, method steps 250 and 260 are carried out first, whereby in order to carry out method step 260, it is not necessary to remove parts of the outer insulation 130, which is only formed later in this method. Only in method step 240 does the outer insulation 130 then form a sheath. Depending on the embodiment of this procedure, it may be necessary to free the previously electrically contacted electrodes 140 from the subsequently formed outer insulation 130. Such method steps are familiar to a person skilled in the art.

### REFERENCE SIGNS

100 Catheter
105 Catheter body
110 Inner liner
120 Flexible circuit board
121 Electrical conductor
122 Contact surface
130 Outer insulation
140 Electrode
145 Contact opening with contact means
150 Temperature sensor
200 Method for producing a catheter
210 Providing an inner liner
220 Providing a flexible circuit board with temperature sensor
230 Arranging
240 Sheathing
250 Providing an electrode
260 Electrical contact of the electrode

What is claimed is:

1. A catheter with a temperature sensor system comprising
a catheter body comprising a hollow cylindrical inner liner having an inner liner outer surface, the inner liner extending in a longitudinal direction from a proximal catheter body end to a distal catheter body end of the catheter body,
an outer insulation having an inner insulation surface, the outer insulation coaxially surrounding the inner liner, and,
a flexible circuit board comprising a plurality of electrical conductors, wherein the flexible circuit board is arranged between the inner liner outer surface and the inner insulation surface and,
at least one electrode at the distal catheter body end, wherein the electrode is electrically conductively connected to at least one of the electrical conductors, wherein the flexible circuit board comprises at least one temperature sensor, wherein the temperature sensor is electrically conductively connected to at least one of the electrical conductors.

2. The catheter according to claim 1, wherein the temperature sensor is a printed temperature sensor.

3. The catheter according to claim 1, wherein the flexible circuit board is arranged at least partially in a spiral shape in turns around the inner liner.

4. The catheter according to claim 1, wherein the flexible circuit board is fixed between the inner liner and the outer insulation.

5. The catheter according to claim 1, wherein the inner liner and the outer insulation are at least partially integrally connected to one another.

6. The catheter according to claim 1, wherein the temperature sensor is designed as a meandering electrical conductor track.

7. The catheter according to claim 6, wherein the meandering electrical conductor track comprises at least one metal selected from the group consisting of silver, platinum, copper and gold.

8. The catheter according to claim 1, wherein the temperature sensor is designed as a thermistor.

9. The catheter according to claim 8, wherein the thermistor is a printed thermistor.

10. The catheter according to claim 8, wherein the thermistor comprises at least one material selected from the group consisting of poly-3,4-ethylenedioxythiophene, polydimethylsiloxane, carbon nanotubes, graphene oxide, silicon carbide and graphene.

11. The catheter according to claim 1, wherein the temperature sensor has a radial extension which is less than a radial extension of the outer insulation.

12. The catheter according to claim 1, wherein the at least one temperature sensor is arranged at the same axial height of the catheter as the at least one electrode.

13. A method for producing a catheter with a temperature sensor system according to any of claim 1, comprising the method steps of:
a) providing a hollow cylindrical inner liner with a radially outer inner liner outer surface;
b) providing a flexible circuit board comprising a plurality of electrical conductors and comprising at least one temperature sensor, wherein the temperature sensor is electrically conductively connected to at least one of the electrical conductors;
c) arranging the flexible circuit board on the inner liner outer surface;
d) sheathing the inner liner and the flexible circuit board with an outer insulation;
e) providing at least one electrode;
f) electrically contacting the at least one electrode with at least one of the electrical conductors of the flexible circuit board.

14. The method according to claim 13, wherein the sheathing of the inner liner in method step d) comprises pulling at least one hollow cylindrical polymer tube onto the inner liner and the flexible circuit board, which tube, after being pulled on, undergoes a radial contraction by heating in order to form the outer insulation.

15. The method according to claim 13, wherein, for electrically contacting the electrode in method step f), at least one contact opening is created in the outer insulation in order to selectively electrically contact the electrode with at least one electrical conductor of the flexible circuit board that is accessible via the contact opening.

16. The catheter according to claim 2, wherein the flexible circuit board is arranged at least partially in a spiral shape in turns around the inner liner.

17. The catheter according to claim 2, wherein the flexible circuit board is fixed between the inner liner and the outer insulation.

18. The catheter according to claim 3, wherein the flexible circuit board is fixed between the inner liner and the outer insulation.

19. The catheter according to claim 2, wherein the inner liner and the outer insulation are at least partially integrally connected to one another.

20. The catheter according to claim 3, wherein the inner liner and the outer insulation are at least partially integrally connected to one another.
